(19) United States
(12) Patent Application Publication
(10) Pub. No.: US 2026/0123649 A1
(43) Pub. Date: May 7, 2026

(54) COMPOSITIONS AND METHODS FOR NUTRACEUTICAL GUMMY

(71) Applicant: NOON WORLD INC., Brooklyn, NY (US)

(72) Inventors: Julie Sok Lin Hwang, Toronto (CA); Jane Kate Wong, Brooklyn, NY (US); Christine Tse-Mun Wong, North York (CA)

(21) Appl. No.: 19/376,049

(22) Filed: Oct. 31, 2025

Related U.S. Application Data
(60) Provisional application No. 63/714,525, filed on Oct. 31, 2024.

(51) Int. Cl. A23G 3/54; A23G 3/36; A23G 3/42; A23G 3/48; A61K 9/00; A61K 31/05; A61K 31/122; A61K 31/197; A61K 31/198; A61K 31/4375; A61K 31/4415; A61K 31/519; A61K 31/522; A61K 31/593; A61K 31/714; A61K 33/06; A61K 36/068; A61K 36/07; A61K 36/074; A61K 36/185; A61K 36/258; A61K 36/28; A61K 36/35; A61K 36/41; A61K 36/79; A61K 36/81; A61K 36/815; A61K 36/82; A61K 36/88; A61K 36/9062; A61K 47/36

(52) U.S. Cl. CPC A23G 3/54; A23G 3/368; A23G 3/42; A23G 3/48; A61K 9/0056; A61K 31/05; A61K 31/122; A61K 31/197; A61K 31/198; A61K 31/4375; A61K 31/4415; A61K 31/519; A61K 31/522; A61K 31/593; A61K 31/714; A61K 33/06; A61K 36/068; A61K 36/07; A61K 36/074; A61K 36/185; A61K 36/258; A61K 36/28; A61K 36/35; A61K 36/41; A61K 36/79; A61K 36/81; A61K 36/815; A61K 36/82; A61K 36/88; A61K 36/9062; A61K 47/36

(57) ABSTRACT

A nutraceutical gummy includes a dual-layer configuration that has an inner core and, distinct from the inner core, a casing shell fully encasing the inner core. The inner core has a core composition including, by weight percentage, 55-85% of a syrup, 10-20% of water, 1-7% of stevia leaf extract, and 1-5% of pectin. The casing shell has a shell composition including, by weight percentage, 55-85% of a syrup, 10-20% of water, 1-7% of stevia leaf extract, and 1-5% of pectin.

### TECHNICAL FIELD

The present disclosure pertains to nutraceutical gummy formulations and their structure and, more specifically, to a dual-layer pectin-based gummy that includes nutraceutical agents for enhanced functional benefits.

### BACKGROUND

Despite increasing consumer demand for functional supplements aimed at managing stress and enhancing cognition, existing formulations often lack optimal bioavailability, stability, or consumer compliance. Traditional oral supplements, including capsules and powders, may have low absorption rates and require multiple doses to achieve efficacy. Additionally, the taste, texture, and overall consumption experience may deter consistent use.

### SUMMARY

A nutraceutical gummy according to an example of the present disclosure includes a dual-layer configuration that has an inner core and, distinct from the inner core, a casing shell fully encasing the inner core. The inner core has a core composition including, by weight percentage, 55-85% of a syrup, 10-20% of water, 1-7% of stevia leaf extract, and 1-5% of pectin. The casing shell has a shell composition including, by weight percentage, 55-85% of a syrup, 10-20% of water, 1-7% of stevia leaf extract, and 1-5% of pectin.

In a further embodiment of the preceding embodiment, the core composition further includes 0.01-1% of L-malic acid, 0.01-1% of sodium citrate, 0.01-1% of natural flavors, 0.01-1% of natural colors, and 0.01-1% of carnauba wax, and the shell composition further includes 0.01-1% of L-malic acid, 0.01-1% of sodium citrate, 0.01-1% of carnauba wax, 0.01-1% of natural flavors, and 0-1% of natural colors.

In a further embodiment of any of the preceding or succeeding embodiments, the core composition further includes 0.1-1% of L-theanine.

In a further embodiment of any of the preceding or succeeding embodiments, the core composition further includes gamma-aminobutyric acid.

In a further embodiment of any of the preceding or succeeding embodiments, the core composition further includes gamma-aminobutyric acid and L-theanine, and a ratio, by weight, of the L-theanine to the gamma-aminobutyric acid is from 5:1 to 1:5 for relaxation or sleep.

In a further embodiment of any of the preceding or succeeding embodiments, the core composition further includes 0.1-3% of a constituent selected from the group consisting of Camellia sinensis, Hericium erinaceus, Cordyceps sinensis, Rhodiola rosea, Lycium barbarum, vitamin B6, vitamin B9, vitamin B12, vitamin D3, Ganodema lucidum extract, and combinations thereof.

In a further embodiment of any of the preceding or succeeding embodiments, the core composition further includes 0.1-1% of a constituent selected from the group consisting of Withania somnifera, Inonotus obliquus, Grifola frondosa, Coriolis versicolor, Lentinula edodes, Passiflora incarnata, Valeriana officinalis, Tremella fuciformis, Wolfporia extensa, Matricaria chamomilla, magnesium glycinate, caffeine, creatine, Alpinia galanga extract, Panax ginseng extract, Schisandra chinensis extract, theobromine, Crocus sativus (saffron) extract, magnesium L-theonate, Conenzyme Q10, N-acetylcysteine, berberine, Resveratrol, Pterostilbene, and combinations thereof.

In a further embodiment of any of the preceding or succeeding embodiments, the core composition further includes 0.1-1% Crocus sativus (Saffron) 0.1-1% Panax Ginseng, 0.1-1% Alpinia Galanga, and 0.1-1% Schisandra chinensis for energy.

In a further embodiment of any of the preceding or succeeding embodiments, the core composition further includes 0.5-1% of Camellia sinensis extract and 0.1-1% of L-theanine for focus and energy.

In a further embodiment of any of the preceding or succeeding embodiments, the core composition further includes 0.1-3% of Camellia sinensis and 0.1-1% of Cordyceps sinensis for energy and endurance.

In a further embodiment of any of the preceding or succeeding embodiments, the core composition further includes 0.1-1% of Cordyceps sinensis and 0.1-1% Rhodiola rosea for stamina.

In a further embodiment of any of the preceding or succeeding embodiments, the core composition further includes 0.1-1% of caffeine and 0.1-1% of L-theanine for focus and energy.

In a further embodiment of any of the preceding or succeeding embodiments, the core composition further includes 0.1-1% of caffeine and 0.1-1% theobromine for focus and energy.

In a further embodiment of any of the preceding or succeeding embodiments, the core composition further includes 0.1-1% of caffeine and 0.1-1% creatine for focus and energy.

In a further embodiment of any of the preceding or succeeding embodiments, the core composition further includes 0.1-1% of caffeine and 0.1-1% Alpinia galanga for energy.

In a further embodiment of any of the preceding or succeeding embodiments, the core composition further includes 0.1-1% gamma-aminobutyric acid and 0.1-1% of Passiflora incarnata for relaxation and sleep, or 0.1-1% of Valeriana officinalis and 0.1-1% L-theanine for sleep.

In a further embodiment of any of the preceding or succeeding embodiments, the core composition further includes 0.1-1% of magnesium and 0.1-1% of L-theanine for relaxation or focus.

In a further embodiment of any of the preceding or succeeding embodiments, the core composition further includes at least one of:
0.5-1% of Camellia sinensis and 0.1-1% of L-theanine,
0.1-3% of Camellia sinensis and 0.1-1% of Cordyceps sinensis,
0.1-1% of Cordyceps sinensis and 0.1-1% of Rhodiola rosea
0.1-1% of caffeine and 0.1-1% of L-theanine,
0.1-1% of caffeine and 0.1-1% of theobromine,
0.1-1% of caffeine and 0.1-1% of creatine,
0.1-1% of caffeine and 0.1-1% of Alpinia galanga,
0.1-1% of gamma-aminobutyric acid and 0.1-1% of L-theanine,
0.1-1% of gamma-aminobutyric acid and 0.1-1% of Passiflora incarnata,
0.1-1% of Valeriana officinalis and 0.1-1% of Passiflora incarnata,
0.1-1% of Valeriana officinalis and 0.1-1% of L-theanine, or
0.1-1% of magnesium and 0.1-1% of L-theanine.

A method of administration of a nutraceutical gummy according to an example of the present disclosure includes providing a serving of the nutraceutical gummy for oral ingestion. The nutraceutical gummy has a dual-layer configuration including an inner core and, distinct from the inner core, a casing shell fully encasing the inner core, the inner core has a core composition in accordance with any of the preceding embodiments. The casing shell has a shell composition according to any of the preceding embodiments.

In a further embodiment of the preceding embodiment, the oral ingestion includes mastication by chewing, at least a portion of the core composition absorbed through buccal and sublingual mucosa.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1A illustrates an example of a nutraceutical gummy.

FIG. 1B is a top view of the nutraceutical gummy.

FIG. 1C is a side view of the nutraceutical gummy.

### DETAILED DESCRIPTION

The present disclosure pertains to nutraceutical formulations and their delivery systems. Examples according to the present disclosure include a dual-layer, pectin-based gummy composition containing bioactive ingredients in distinct ingredient profiles in each of the layers. Bioactive ingredients include but are not limited to bioactive molecules, such as but not limited to L-theanine, nootropics, adaptogens, herbal extracts, such as but not limited to mushroom extracts and plant extracts, vitamins, minerals, amino acids, probiotics, essential fatty acids, and hydrolyzed collagen peptides. This dual-layer format in combination with the nutraceutical formulations offer an innovative solution in the fields such as, but not limited to cognitive enhancement, energy improvement, relaxation, mood enhancement, stress reduction, sexual vitality, muscle recovery, multivitamin nutrition, fertility support and hormonal balance.

Examples according to the present disclosure harnesses the pectin-based dual-layered matrix not only for improved palatability and compliance, but also to enhance ingredient stability and bioavailability, leveraging a dual-layered gummy for precise ingredient encapsulation. This dual-layer, pectin-based gummy formulation delivers bioactive ingredients in a stable, consumer-friendly format. The formulation utilizes a liquid, semi-solid, or solid core section containing synergistic ingredients for functions including but not limited to enhanced cognitive function and stress reduction, enclosed by a pectin-based casing section shell providing structural integrity, moisture stability, and a vegan-friendly delivery mechanism. Encapsulated within the gummy are carefully chosen ratios, amounts, and percentages of active ingredients, such as various herbal extracts, nootropics, adaptogens, functional mushrooms, and nutrients such as vitamins, minerals, and electrolytes-enabling more precise dosing and reliable efficacy.

Embodiments of the present disclosure provide compositions or mechanisms for oral administration of bioactive ingredients via a functional nutraceutical dual-layer pectin-based gummy composition.

For example, in some embodiments, the nutraceutical dual-layer pectin gummy comprises L-theanine and/or gamma-aminobutyric acid (“GABA”), with or without additional bioactive components, in combination with a nutraceutical-acceptable carrier. In various embodiments, the ratio of L-theanine to GABA is approximately 5:1 to 1:5. In various embodiments, the nutraceutical dual-layer pectin-based gummy composition may provide L-theanine in an approximate range of about 5 mg to 300 mg per serving; GABA may be provided in an approximate range from about 0 mg to about 300 mg per serving. In some embodiments, L-theanine may comprise a range of about 0.1% to 10% by weight of the composition; and GABA may comprise up to approximately 10% by weight of the composition.

In various embodiments, the ratio of L-theanine to GABA may range from 5:1 to 1:1, supporting calmness without drowsiness, reduction in cortisol levels to mitigate effects of stress and anxiety, balancing of mood through neurotransmitter regulation, and enhancing cognitive functions like focus and attention.

In additional embodiments, the ratio of L-theanine to GABA may range from 1:1 to 1:5, supporting restorative rest, decreased sleep latency, and improved overall sleep quality.

In various other embodiments, L-theanine may be included without the presence of GABA, supporting a calm and alert wakefulness that supports good mood and lifted energy without drowsiness through the activation of alpha brain waves and reduction of cortisol levels.

Specific ratios, amounts, and percentages of active ingredients, and their combinations with other ingredients have demonstrated beneficial outcomes in the mind and body when combined into this unique dual-layer gummy delivery system.

### 1. Gummy Structure

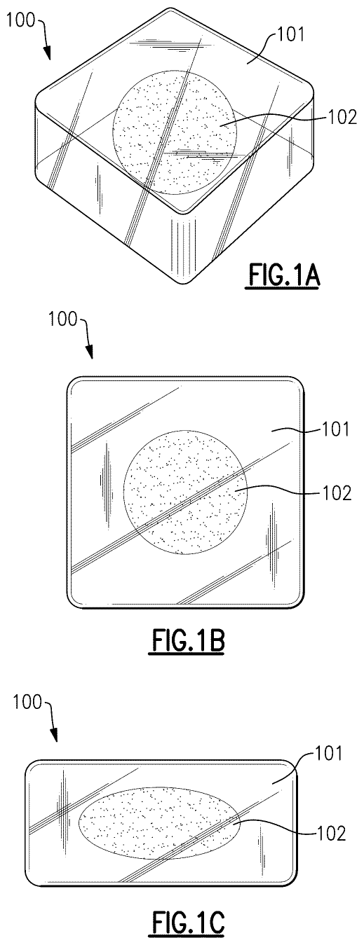

FIG. 1A illustrates an example of a nutraceutical gummy 100 in accordance with embodiments of the present disclosure. FIG. 1B shows a top view, and FIG. 1C shows a side view. The nutraceutical gummy 100 has a distinct dual-layer configuration. The dual layer configuration includes a casing shell layer 101 and, distinct from the casing shell layer 101, an inner core layer 102. For instance, the casing shell layer 101 and the inner core layer 102 are visibly distinct without magnification, such as by differing colors or differing shades of a color. In the example, the casing shell layer 101 completely encases the inner core layer 102, which physically protects the inner core layer 102 and inhibits ingress of gases and moisture. The inner core layer 102 is formulated of core ingredients, described in further examples below. The casing shell layer 101 is formulated of casing ingredients, also described in further detail below. The formulations of the casing shell layer 101 and the inner core layer 102 differ in at least one ingredient, but more typically differ in multiple ingredients.

### 1A. Core Active Ingredients and Combinations

In the compositions herein, ratios (e.g., 10:1) are given for each herbal extract. Use of ratios is common and accepted in the art of describing extracts and is well-known in the art that a ratio represents the concentration of the ingredient. For example, a ratio of 10:1 indicates a ratio of 10 g of starting material that has been concentrated into 1 g of extract. Core active ingredients and combinations are formulated specifically for distinct functional benefits, with consideration for the ability of active ingredients to be absorbed in the mouth and the gastrointestinal (GI) tract, for combined faster and slower absorption, respectively. One example of a core active ingredient is the bioactive amino acid L-theanine, found in natural sources such as green tea and black tea. L-theanine can be effectively absorbed in both the mouth and GI tract, and has been demonstrated in clinical, randomized control trials to have anxiolytic and stress-relief effects, indicated by the reduction of stress marker s-IgA and decreased heart rate. L-theanine modulates alpha-wave activity, promoting relaxation without sedation, and enhancing attention when combined with stimulants such as caffeine. This ingredient also positively influences glutamate and dopamine neurotransmission, contributing to improved mood and cognitive clarity.

In embodiments, core active ingredients such as L-theanine are combined with other nutraceutical agents-including but not limited to bioactive molecules, nootropics, adaptogens, herbal extracts, mushroom extracts, plant extracts, vitamins, minerals, amino acids, probiotics, essential fatty acids, and hydrolyzed collagen peptides—to target functional benefits.

Pertaining to the targeted function of generating increased cognition, alertness, memory, focus and energy: in one example, the core layer 102 has a composition, by weight percentage, of 55-85% of a syrup (a “syrup” as used herein refers to a sugar or sugar alcohol, such as maltitol, erythritol, etc.), 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors (e.g., compounds derived from a natural source, such as a fruit, a vegetable, or a spice), 0.01-1% of natural color (e.g., pigments derived from natural sources, such as plants, minerals, and animals to add or enhance color, as opposed to synthetic dyes), and 0.01-1% of carnauba wax, 0.5-1% of Camellia sinensis Extract (10:1), 0.1-1% of Hericium erinaceus Extract (10:1), 0.1-1% of Cordyceps sinensis Extract (10:1), 0.1-1% of Rhodiola rosea Extract (10:1), 0.1-1% of Lycium barbarum Extract (10:1), 0.1-1% of L-theanine, 0.03-1% of Vitamin B6 (Pyridoxine), 0.0033-1% of Vitamin B9 (Folate), 0.033-1% of Vitamin B12 (Methylcobalamin), 0.0003-1% of Vitamin D3 (Cholecalciferol).

In another particular example, pertaining to the function of targeted anxiolytic and adaptogenic stress relief, the inner core layer 102 has a composition, by weight percentage, of 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-1% of L-theanine, 0.1-1% of Y-aminobutyric acid (GABA), 0.1-1% of Withania somnifera Extract (10:1), 0.1-1% of Rhodiola rosea Extract (10:1), 0.1-1% of Ganoderma lucidum Extract (10:1), 0.1-1% of Cordyceps sinensis Extract (10:1), 0.1-1% of Inonotus obliquus Extract (10:1), 0.1-1% of Hericium erinaceus Extract (10:1), 0.1-1% of Grifola frondosa Extract (10:1), 0.1-1% of Coriolus versicolor Extract (10:1), 0.1-1% of Lentinula edodes Extract (10:1), 0.1-1% of Passiflora incarnata Extract (10:1).

In another particular example, pertaining to the targeted functions of improved sleep quality and physical rejuvenation and recovery: the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-1% of L-theanine, 0.1-1% of Y-aminobutyric acid (GABA), 0.1-1% of Withania somnifera Extract (10:1), 0.1-1% of Passiflora incarnata Extract (10:1), 0.1-1% of Valeriana officinalis Extract, 0.1-1% of Ganoderma lucidum Extract (10:1), 0.1-1% of Tremella fuciformis Extract (10:1), 0.1-1% of Wolfiporia extensa Extract (10:1), 0.1-1% of Matricaria chamomilla Extract (10:1), 0.01-1% of Magnesium glycinate.

In another particular example, pertaining to the targeted functions of improved wakefulness and energy: the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-1% of L-theanine, 0.1-1% of Caffeine, 0.1-1% of Creatine, 0.1-1% of Alpinia Galanga Extract (10:1), 0.1-1% of Cordyceps sinensis Extract (10:1), 0.1-1% of Panax Ginseng Extract, 0.1-1% of Schisandra chinensis Extract (10:1), 0.033-1% of Vitamin B12 (Methylcobalamin), 0-1% of Theobromine.

In another particular example, pertaining to the targeted functions of improved mood and energy: the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-1% of L-theanine, 0.1-1% of Caffeine, 0.0-1% of Creatine, 0.1-1% of Alpinia Galanga Extract (10:1), 0.1-1% of Cordyceps sinensis Extract (10:1), 0.1-1% of Panax Ginseng Extract, 0.1-1% of Schisandra chinensis Extract (10:1), 0.033-1% of Vitamin B12 (Methylcobalamin), 0-1% of Theobromine, 0-1% of Theobroma cacao seed Extract (10:1), 0-1% of Rhodiola rosea Extract (10:1), 0-1% Withania somnifera Extract (10:1), 0-1% of Schisandra chinensis, 0-1% of Ganoderma lucidum, 0-1% of Calendula officinalis Extract (10:1), 0-1% of Echinacea angustifolia Extract (10:1), and 0-1% of Echinacea purpurea Extract (10:1).

In another particular example, pertaining to the targeted functions of improved libido, blood circulation, and energy: the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-1% of Crocus sativus (Saffron) Extract, 0.1-3% Camellia sinensis Extract, 0.1-5% of Theobromine, 0.1-1% of Cordyceps sinensis Extract (10:1), 0.1-1% of Ganoderma lucidum Extract (10:1), 0.1-1% of Lentinula edodes Extract (10:1), 0.1-2% of Magnesium L-Threonate, 0.1-2% of Magnesium Glycinate.

In another particular example, pertaining to the function of targeted multivitamin nutrition, immunity, or muscle recovery, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0-35% of Magnesium, 0-1% of Zinc, 0-1% of Iron, 0-1% of Calcium, 0.0003-1% of Vitamin D3, 0-2% of Vitamin K2, 0-2% of Vitamin C, 0-1% of Vitamin B1, 0-1% of Vitamin B2, 0-1% of Vitamin B3, 0-1% of Vitamin B5, 0-1% of Vitamin B6, 0-1% of Vitamin B7, 0-1% of Vitamin B8, 0-1% of Vitamin B12, 0-1% of Citicoline, 0-1% of Inositol, 0-2% of Potassium, 0-1% of Omega-3, 0-3% of Omega-6, 0-3% of Omega-9, 0-3% of Collagen.

In another particular example, pertaining to the function of targeted fertility support and hormonal balance, pain management, and skin health, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0-1% of Folate, 0-2% of Coenzyme Q10, 0-2% of N-acetylcysteine (NAC), 0-2% of L-Acetyl Carnitine (LAC), 0-2% of R-Lipoic Acid, 0-2% of Vitex-Agnus Castus Extract (10:1), 0-2% of Inositol, 0-2% of Arginine, 0-1% of Vitamin D3, 0-1% of Magnesium, 0-5% of Zingiber officinale, 0-1% of Withania somnifera Extract (10:1), 0-1% of Tremella fuciformis Extract (10:1), 0-1% of Grifola frondosa Extract (10:1), 0-1% of Lentinula edodes Extract (10:1), 0-1% Silybum marianum Extract (10-1), 0-3% of Collagen.

In another particular example, pertaining to the function of targeted improved metabolism, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0-40% Apple cider vinegar Extract, 0-3% Camellia sinensis Extract (10:1), 0-2% of L-Acetyl Carnitine (LAC), 0-2% of R-Lipoic Acid, 0-3% of Inositol, 0-2% of Arginine, 0-1% Vitamin B12 (Methylcobalamin), 0-1% Vitamin B6 (Pyridoxine), 0-1% Vitamin B3, 0-5% of Zingiber officinale, 0-1% of Gynostemma pentaphyllum (10:1), 0-1% of Gymnema sylvestre Extract (10:1), 0-0.001% of Chromium, 0-2% of Berberine, 0-0.1% of Lagerstroemia speciosa leaf Extract (10:1), 0-0.1% of Grains of Paradise (Aframomum melegueta) (10:1).

In another particular example, pertaining to the function of targeted pain, low mood, fatigue, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.0003-1% of Vitamin D3, 0-1% of Vitamin B1 (Thiamine), 0.03-1% of Vitamin B6 (Pyridoxine), 0.0033-1% of Vitamin B9 (Folate), 0.033-1% of Vitamin B12 (Methylcobalamin), 0-1% of Vitamin C, 0-1% of Curcumin, 0-1% of Piperine, 0-1% of Panax Ginseng (10:1), 0-1% of Cistanche tubulosa Extract (10:1), 0-1% Stinging Nettle (Urtica dioica) Extract (10:1), 0-1% of Griffonia simplicifolia Extract (10:1), 0-1% of Crocus sativus (Saffron) Extract (10:1), 0-1% of Magnesium, 0-1% of Hericium erinaceus Extract (10:1), 0-1% of Ganoderma lucidum Extract (10:1), 0-1% of Cordyceps sinensis Extract (10:1), 0-1% of Inonotus obliquus Extract (10:1).

In another particular example, pertaining to the targeted function of improved recovery and mental clarity: the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0-1% of Crocus sativus Extract, 0-3% Camellia sinensis Extract, 0.1-7% of Theobromine (10:1), 0-3% of Cordyceps sinensis Extract (10:1), 0.1-2% of Magnesium L-Threonate, 0-1% of Vitamin B3, 0-1% of Hericium erinaceus Extract (10:1), 0-1% of Ganoderma lucidum Extract (10:1).

In another particular example, pertaining to the function of targeted longevity, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0.1-2% of CoQ10 (Ubiquinol), 0.1-5% of NAC (N-Acetyl-Cysteine), 0.1-2% of Resveratrol, 0.1-2% of Pterostilbene, 0.1-5% of Berberine, 0-4% of Nicotinamide Mononucleotide (NMN), 0-2% of R-Alpha Lipoic Acid (R-LA), 0-2% of Fisetin, 0-3% of Milk Thistle (Silybum marianum), 0-3% of Quercetin, 0-0.01% of Selenium, 0-3% of Vitamin C, 0-3% of Vitamin B3 (Niacinamide), 0-1% of Hericium erinaceus Extract (10:1), 0-1% of Ganoderma lucidum Extract (10:1), 0-1% of Inonotus obliquus Extract (10:1).

In another particular example, pertaining to the function of targeted anxiolytic, adaptogen and immunity, the inner core layer 102 has a composition, by weight percentage, 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of natural flavors, 0.01-1% of natural color, and 0.01-1% of carnauba wax, 0-1% of Ganoderma lucidum Extract (10:1), 0-1% of Cordyceps sinensis Extract (10:1), 0-1% of Inonotus obliquus Extract (10:1), 0-1% of Hericium erinaceus Extract (10:1), 0-1% of Grifola frondosa Extract (10:1), 0-1% of Coriolus versicolor Extract (10:1), 0-1% of Lentinula edodes Extract (10:1), 0-1% of Tremella fuciformis Extract (10:1), 0-1% of Wolfiporia extensa Extract (10:1), 0-1% of Trametes versicolor Extract (10:1), 0-1% of Pleurotus ostreatus Extract (10:1), 0-1% of Flammulina velutipes Extract (10:1), 0-1% of Agaricus blazei Extract (10:1).

The example core compositions above are also summarized in the table below.

| Ingredient | Example | | | | | | |
|---|---|---|---|---|---|---|---|
| (wt. %) | 1 | 2 | 3 | 4 | 5 | 6 | 7 |
| Syrup | 55-85 | 55-85 | 55-85 | 55-85 | 55-85 | 55-85 | 55-85 |
| Maltodextrin | 0-25 | 0-25 | 0-25 | 0-25 | 0-25 | 0-25 | 0-25 |
| (optional) | | | | | | | |
| Water | 10-20 | 10-20 | 10-20 | 10-20 | 10-20 | 10-20 | 10-20 |
| Stevia leaf | 1-7 | 1-7 | 1-7 | 1-7 | 1-7 | 1-7 | 1-7 |
| extract | | | | | | | |
| Pectin | 1-5 | 1-5 | 1-5 | 1-5 | 1-5 | 1-5 | 1-5 |
| L-malic acid | 0-1 | 0-1 | 0-1 | 0-1 | 0-1 | 0-1 | 0-1 |
| Sodium citrate | 0.01-1 | 0.01-1 | 0.01-1 | 0.01-1 | 0.01-1 | 0.01-1 | 0.01-1 |
| Natural flavors | 0.01-1 | 0.01-1 | 0.01-1 | 0.01-1 | 0.01-1 | 0.01-1 | 0.01-1 |
| Natural color | 0.01-1 | 0.01-1 | 0.01-1 | 0.01-1 | 0.01-1 | 0.01-1 | 0.01-1 |
| Carnauba wax | 0.01-1 | 0.01-1 | 0.01-1 | 0.01-1 | 0.01-1 | 0.01-1 | 0.01-1 |
| Camellia | 0.5-1 | 0 | 0 | 0 | 0 | 0.1-3 | 0 |
| sinensis Extract | | | | | | | |
| Hericium | 0.1-1 | 0.1-1 | 0 | 0 | 0 | 0 | 0 |
| erinaceus | | | | | | | |
| Extract | | | | | | | |
| Cordyceps | 0.1-1 | 0.1-1 | 0 | 0.1-1 | 0.1-1 | 0.1-1 | 0 |
| sinensis Extract | | | | | | | |
| Rhodiola rosea | 0.1-1 | 0.1-1 | 0 | 0 | 0-1 | 0 | 0 |
| Extract | | | | | | | |
| Lycium | 0.1-1 | 0 | 0 | 0 | 0 | 0 | 0 |
| barbarum | | | | | | | |
| Extract | | | | | | | |
| L-theanine | 0.1-1 | 0.1-1 | 0.1-1 | 0.1-1 | 0.1-1 | 0 | 0 |
| Vitamin B6 | 0.03-1 | 0 | 0 | 0 | 0 | 0 | 0-1 |
| (Pyridoxine) | | | | | | | |
| Vitamin B9 | 0.0033-1 | 0 | 0 | 0 | 0 | 0 | 0 |
| (Folate) | | | | | | | |
| Vitamin B12 | 0.0033-1 | 0 | 0 | 0.0033-1 | 0.0033-1 | 0 | 0-1 |
| (Methylcobalamin) | | | | | | | |
| Vitamin D3 | 0.0003-1 | 0 | 0 | 0 | 0 | 0 | 0.0003-1 |
| (Cholecalciferol) | | | | | | | |
| γ-aminobutyric | 0 | 0.1-1 | 0.1-1 | 0 | 0 | 0 | 0 |
| acid (GABA) | | | | | | | |
| Withania | 0 | 0.1-1 | 0.1-1 | 0 | 0-1 | 0 | 0 |
| somnifera | | | | | | | |
| Extract | | | | | | | |
| Ganoderma | 0 | 0.1-1 | 0.1-1 | 0 | 0-1 | 0.1-1 | 0 |
| lucidum | | | | | | | |
| Extract | | | | | | | |
| Inonotus | 0 | 0.1-1 | 0 | 0 | 0 | 0 | 0 |
| obliquus | | | | | | | |
| Extract | | | | | | | |
| Grifola | 0 | 0.1-1 | 0 | 0 | 0 | 0 | 0 |
| frondosa | | | | | | | |
| Extract | | | | | | | |
| Coriolus | 0 | 0.1-1 | 0 | 0 | 0 | 0 | 0 |
| versicolor | | | | | | | |
| Extract | | | | | | | |
| Lentinula | 0 | 0.1-1 | 0 | 0 | 0 | 0.1-1 | 0 |
| edodes Extract | | | | | | | |
| Passiflora | 0 | 0.1-1 | 0.1-1 | 0 | 0 | 0 | 0 |
| incarnata | | | | | | | |
| Extract | | | | | | | |
| Valeriana | 0 | 0 | 0.1-1 | 0 | 0 | 0 | 0 |
| officinalis | | | | | | | |
| Extract | | | | | | | |
| Tremella | 0 | 0 | 0.1-1 | 0 | 0 | 0 | 0 |
| fuciformis | | | | | | | |
| Extract | | | | | | | |
| Wolfiporia | 0 | 0 | 0.1-1 | 0 | 0 | 0 | 0 |
| extensa Extract | | | | | | | |
| Matricaria | 0 | 0 | 0.1-1 | 0 | 0 | 0 | 0 |
| chamomilla | | | | | | | |
| Extract | | | | | | | |
| Magnesium | 0 | 0 | 0.01-1 | 0 | 0 | 0.1-2 | 0 |
| glycinate | | | | | | | |
| Caffeine | 0 | 0 | 0 | 0.1-1 | 0.1-1 | 0 | 0 |
| Creatine | 0 | 0 | 0 | 0.1-1 | 0.0-1 | 0 | 0 |
| Alpinia | 0 | 0 | 0 | 0.1-1 | 0.1-1 | 0 | 0 |
| Galanga | | | | | | | |
| Extract | | | | | | | |
| Panax Ginseng | 0 | 0 | 0 | 0.1-1 | 0.0-1 | 0 | 0 |
| Extract | | | | | | | |
| Schisandra | 0 | 0 | 0 | 0.1-1 | 0.1-1 | 0 | 0 |
| chinensis | | | | | | | |
| Extract | | | | | | | |
| Theobromine | 0 | 0 | 0 | 0-1 | 0-1 | 0.1-5 | 0 |
| Theobroma | 0 | 0 | 0 | 0 | 0-1 | 0 | 0 |
| cacao seed | | | | | | | |
| Extract | | | | | | | |
| Calendula | 0 | 0 | 0 | 0 | 0-1 | 0 | 0 |
| officinalis | | | | | | | |
| Extract | | | | | | | |
| Echinacea | 0 | 0 | 0 | 0 | 0-1 | 0 | 0 |
| angustifolia | | | | | | | |
| Extract | | | | | | | |
| Echinacea | 0 | 0 | 0 | 0 | 0-1 | 0 | 0 |
| purpurea | | | | | | | |
| Extract | | | | | | | |
| Crocus sativus | 0 | 0 | 0 | 0 | 0 | 0.1-1 | 0 |
| (Saffron) | | | | | | | |
| Extract | | | | | | | |
| Magnesium L- | 0 | 0 | 0 | 0 | 0 | 0.1-2 | 0 |
| Threonate | | | | | | | |
| Magnesium | 0 | 0 | 0 | 0 | 0 | 0 | 0-35 |
| Zinc | 0 | 0 | 0 | 0 | 0 | 0 | 0-1 |
| Iron | 0 | 0 | 0 | 0 | 0 | 0 | 0-1 |
| Calcium | 0 | 0 | 0 | 0 | 0 | 0 | 0-1 |
| Vitamin K2 | 0 | 0 | 0 | 0 | 0 | 0 | 0-2 |
| Vitamin C | 0 | 0 | 0 | 0 | 0 | 0 | 0-2 |
| Vitamin B1 | 0 | 0 | 0 | 0 | 0 | 0 | 0-1 |
| (Thiamine) | | | | | | | |
| Vitamin B2 | 0 | 0 | 0 | 0 | 0 | 0 | 0-1 |
| Vitamin B3 | 0 | 0 | 0 | 0 | 0 | 0 | 0-1 |
| (Niacinamide) | | | | | | | |
| Vitamin B5 | 0 | 0 | 0 | 0 | 0 | 0 | 0-1 |
| Vitamin B7 | 0 | 0 | 0 | 0 | 0 | 0 | 0-1 |
| Vitamin B8 | 0 | 0 | 0 | 0 | 0 | 0 | 0-1 |
| Citicoline | 0 | 0 | 0 | 0 | 0 | 0 | 0-1 |
| Inositol | 0 | 0 | 0 | 0 | 0 | 0 | 0-1 |
| Potassium | 0 | 0 | 0 | 0 | 0 | 0 | 0-2 |
| Omega-3 | 0 | 0 | 0 | 0 | 0 | 0 | 0-1 |
| Omega-6 | 0 | 0 | 0 | 0 | 0 | 0 | 0-3 |
| Omega-9 | 0 | 0 | 0 | 0 | 0 | 0 | 0-3 |
| Collagen | 0 | 0 | 0 | 0 | 0 | 0 | 0-3 |
| Folate | 0 | 0 | 0 | 0 | 0 | 0 | 0 |
| Coenzyme Q10 | 0 | 0 | 0 | 0 | 0 | 0 | 0 |
| N- | 0 | 0 | 0 | 0 | 0 | 0 | 0 |
| acetylcysteine | | | | | | | |
| (NAC) | | | | | | | |
| L-Acetyl | 0 | 0 | 0 | 0 | 0 | 0 | 0 |
| Carnitine | | | | | | | |
| (LAC) | | | | | | | |
| R-Lipoic Acid | 0 | 0 | 0 | 0 | 0 | 0 | 0 |
| Vitex-Agnus | 0 | 0 | 0 | 0 | 0 | 0 | 0 |
| Castus Extract | | | | | | | |
| Arginine | 0 | 0 | 0 | 0 | 0 | 0 | 0 |
| Zingiber | 0 | 0 | 0 | 0 | 0 | 0 | 0 |
| officinale | | | | | | | |
| Silybum | 0 | 0 | 0 | 0 | 0 | 0 | 0 |
| marianum | | | | | | | |
| Extract | | | | | | | |
| Apple cider | 0 | 0 | 0 | 0 | 0 | 0 | 0 |
| vinegar Extract | | | | | | | |
| Gynostemma | 0 | 0 | 0 | 0 | 0 | 0 | 0 |
| pentaphyllum | | | | | | | |
| Gymnema | 0 | 0 | 0 | 0 | 0 | 0 | 0 |
| sylvestre | | | | | | | |
| Extract | | | | | | | |
| Chromium | 0 | 0 | 0 | 0 | 0 | 0 | 0 |
| Berberine | 0 | 0 | 0 | 0 | 0 | 0 | 0 |
| Lagerstroemia | 0 | 0 | 0 | 0 | 0 | 0 | 0 |
| speciosa leaf | | | | | | | |
| Extract | | | | | | | |
| Grains of | 0 | 0 | 0 | 0 | 0 | 0 | 0 |
| Paradise | | | | | | | |
| Extract | | | | | | | |
| Curcumin | 0 | 0 | 0 | 0 | 0 | 0 | 0 |
| Piperine | 0 | 0 | 0 | 0 | 0 | 0 | 0 |
| Cistanche | 0 | 0 | 0 | 0 | 0 | 0 | 0 |
| tubulosa | | | | | | | |
| Extract | | | | | | | |
| Stinging Nettle | 0 | 0 | 0 | 0 | 0 | 0 | 0 |
| Extract | | | | | | | |
| Griffonia | 0 | 0 | 0 | 0 | 0 | 0 | 0 |
| simplicifolia | | | | | | | |
| Extract | | | | | | | |
| CoQ10 | 0 | 0 | 0 | 0 | 0 | 0 | 0 |
| (Ubiquinol) | | | | | | | |
| NAC (N- | 0 | 0 | 0 | 0 | 0 | 0 | 0 |
| Acetyl- | | | | | | | |
| Cysteine) | | | | | | | |
| Resveratrol | 0 | 0 | 0 | 0 | 0 | 0 | 0 |
| Pterostilbene | 0 | 0 | 0 | 0 | 0 | 0 | 0 |
| Nicotinamide | 0 | 0 | 0 | 0 | 0 | 0 | 0 |
| Mononucleotide | | | | | | | |
| (NMN) | | | | | | | |
| Fisetin | 0 | 0 | 0 | 0 | 0 | 0 | 0 |
| Milk Thistle | 0 | 0 | 0 | 0 | 0 | 0 | 0 |
| Extract | | | | | | | |
| Quercetin | 0 | 0 | 0 | 0 | 0 | 0 | 0 |
| Selenium | 0 | 0 | 0 | 0 | 0 | 0 | 0 |
| Trametes | 0 | 0 | 0 | 0 | 0 | 0 | 0 |
| versicolor | | | | | | | |
| Extract | | | | | | | |
| Pleurotus | 0 | 0 | 0 | 0 | 0 | 0 | 0 |
| ostreatus | | | | | | | |
| Extract | | | | | | | |
| Flammulina | 0 | 0 | 0 | 0 | 0 | 0 | 0 |
| velutipes | | | | | | | |
| Extract | | | | | | | |
| Agaricus blazei | 0 | 0 | 0 | 0 | 0 | 0 | 0 |
| Extract | | | | | | | |

| Ingredient | Example | | | | | |
|---|---|---|---|---|---|---|
| (wt. %) | 8 | 9 | 10 | 11 | 12 | 13 |
| Syrup | 55-85 | 55-85 | 55-85 | 55-85 | 55-85 | 55-85 |
| Maltodextrin | 0-25 | 0-25 | 0-25 | 0-25 | 0-25 | 0-25 |
| (optional) | | | | | | |
| Water | 10-20 | 10-20 | 10-20 | 10-20 | 10-20 | 10-20 |
| Stevia leaf | 1-7 | 1-7 | 1-7 | 1-7 | 1-7 | 1-7 |
| extract | | | | | | |
| Pectin | 1-5 | 1-5 | 1-5 | 1-5 | 1-5 | 1-5 |
| L-malic acid | 0-1 | 0-1 | 0-1 | 0-1 | 0-1 | 0-1 |
| Sodium citrate | 0.01-1 | 0.01-1 | 0.01-1 | 0.01-1 | 0.01-1 | 0.01-1 |
| Natural flavors | 0.01-1 | 0.01-1 | 0.01-1 | 0.01-1 | 0.01-1 | 0.01-1 |
| Natural color | 0.01-1 | 0.01-1 | 0.01-1 | 0.01-1 | 0.01-1 | 0.01-1 |
| Carnauba wax | 0.01-1 | 0.01-1 | 0.01-1 | 0.01-1 | 0.01-1 | 0.01-1 |
| Camellia | 0 | 0-3 | 0 | 0-3 | 0 | 0 |
| sinensis Extract | | | | | | |
| Hericium | 0 | 0 | 0-1 | 0-1 | 0-1 | 0-1 |
| erinaceus | | | | | | |
| Extract | | | | | | |
| Cordyceps | 0 | 0 | 0-1 | 0-3 | 0 | 0-1 |
| sinensis Extract | | | | | | |
| Rhodiola rosea | 0 | 0 | 0 | 0 | 0 | 0 |
| Extract | | | | | | |
| Lycium | 0 | 0 | 0 | 0 | 0 | 0 |
| barbarum | | | | | | |
| Extract | | | | | | |
| L-theanine | 0 | 0 | 0 | 0 | 0 | 0 |
| Vitamin B6 | 0 | 0-1 | 0.03-1 | 0 | 0 | 0 |
| (Pyridoxine) | | | | | | |
| Vitamin B9 | 0-1 | 0 | 0.0033-1 | 0 | 0 | 0 |
| (Folate) | | | | | | |
| Vitamin B12 | 0 | 0-1 | 0.0033-1 | 0 | 0 | 0 |
| (Methylcobalamin) | | | | | | |
| Vitamin D3 | 0-1 | 0 | 0.0003-1 | 0 | 0 | 0 |
| (Cholecalciferol) | | | | | | |
| γ-aminobutyric | 0 | 0 | 0 | 0 | 0 | 0 |
| acid (GABA) | | | | | | |
| Withania | 0-1 | 0 | 0 | 0 | 0 | 0 |
| somnifera | | | | | | |
| Extract | | | | | | |
| Ganoderma | 0-1 | 0 | 0-1 | 0-1 | 0-1 | 0-1 |
| lucidum | | | | | | |
| Extract | | | | | | |
| Inonotus | 0 | 0 | 0-1 | 0 | 0-1 | 0-1 |
| obliquus | | | | | | |
| Extract | | | | | | |
| Grifola | 0-1 | 0 | 0 | 0 | 0 | 0-1 |
| frondosa | | | | | | |
| Extract | | | | | | |
| Coriolus | 0 | 0 | 0 | 0 | 0 | 0-1 |
| versicolor | | | | | | |
| Extract | | | | | | |
| Lentinula | 0-1 | 0 | 0 | 0 | 0 | 0-1 |
| edodes Extract | | | | | | |
| Passiflora | 0 | 0 | 0 | 0 | 0 | 0 |
| incarnata | | | | | | |
| Extract | | | | | | |
| Valeriana | 0 | 0 | 0 | 0 | 0 | 0 |
| officinalis | | | | | | |
| Extract | | | | | | |
| Tremella | 0-1 | 0 | 0 | 0 | 0 | 0-1 |
| fuciformis | | | | | | |
| Extract | | | | | | |
| Wolfiporia | 0 | 0 | 0 | 0 | 0 | 0-1 |
| extensa Extract | | | | | | |
| Matricaria | 0 | 0 | 0 | 0 | 0 | 0 |
| chamomilla | | | | | | |
| Extract | | | | | | |
| Magnesium | 0 | 0 | 0 | 0 | 0 | 0 |
| glycinate | | | | | | |
| Caffeine | 0 | 0 | 0 | 0 | 0 | 0 |
| Creatine | 0 | 0 | 0 | 0 | 0 | 0 |
| Alpinia | 0 | 0 | 0 | 0 | 0 | 0 |
| Galanga | | | | | | |
| Extract | | | | | | |
| Panax Ginseng | 0 | 0 | 0-1 | 0 | 0 | 0 |
| Extract | | | | | | |
| Schisandra | 0 | 0 | 0 | 0 | 0 | 0 |
| chinensis | | | | | | |
| Extract | | | | | | |
| Theobromine | 0 | 0 | 0 | 0.1-7 | 0 | 0 |
| Theobroma | 0 | 0 | 0 | 0 | 0 | 0 |
| cacao seed | | | | | | |
| Extract | | | | | | |
| Calendula | 0 | 0 | 0 | 0 | 0 | 0 |
| officinalis | | | | | | |
| Extract | | | | | | |
| Echinacea | 0 | 0 | 0 | 0 | 0 | 0 |
| angustifolia | | | | | | |
| Extract | | | | | | |
| Echinacea | 0 | 0 | 0 | 0 | 0 | 0 |
| purpurea | | | | | | |
| Extract | | | | | | |
| Crocus sativus | 0 | 0 | 0-1 | 0-1 | 0 | 0 |
| (Saffron) | | | | | | |
| Extract | | | | | | |
| Magnesium L- | 0 | 0 | 0 | 0.1-2 | 0 | 0 |
| Threonate | | | | | | |
| Magnesium | 0-1 | 0 | 0-1 | 0 | 0 | 0 |
| Zinc | 0 | 0 | 0 | 0 | 0 | 0 |
| Iron | 0 | 0 | 0 | 0 | 0 | 0 |
| Calcium | 0 | 0 | 0 | 0 | 0 | 0 |
| Vitamin K2 | 0 | 0 | 0 | 0 | 0 | 0 |
| Vitamin C | 0 | 0 | 0-1 | 0 | 0-3 | 0 |
| Vitamin B1 | 0 | 0 | 0-1 | 0 | 0 | 0 |
| (Thiamine) | | | | | | |
| Vitamin B2 | 0 | 0 | 0 | 0 | 0 | 0 |
| Vitamin B3 | 0 | 0-1 | 0 | 0-1 | 0-3 | 0 |
| (Niacinamide) | | | | | | |
| Vitamin B5 | 0 | 0 | 0 | 0 | 0 | 0 |
| Vitamin B7 | 0 | 0 | 0 | 0 | 0 | 0 |
| Vitamin B8 | 0 | 0 | 0 | 0 | 0 | 0 |
| Citicoline | 0 | 0 | 0 | 0 | 0 | 0 |
| Inositol | 0-2 | 0-3 | 0 | 0 | 0 | 0 |
| Potassium | 0 | 0 | 0 | 0 | 0 | 0 |
| Omega-3 | 0 | 0 | 0 | 0 | 0 | 0 |
| Omega-6 | 0 | 0 | 0 | 0 | 0 | 0 |
| Omega-9 | 0 | 0 | 0 | 0 | 0 | 0 |
| Collagen | 0-3 | 0 | 0 | 0 | 0 | 0 |
| Folate | 0-1 | 0 | 0 | 0 | 0 | 0 |
| Coenzyme Q10 | 0-2 | 0 | 0 | 0 | 0.1-2 | 0 |
| N- | 0-2 | 0 | 0 | 0 | 0.1-5 | 0 |
| acetylcysteine | | | | | | |
| (NAC) | | | | | | |
| L-Acetyl | 0-2 | 0-2 | 0 | 0 | 0 | 0 |
| Carnitine | | | | | | |
| (LAC) | | | | | | |
| R-Lipoic Acid | 0-2 | 0-2 | 0 | 0 | 0-2 | 0 |
| Vitex-Agnus | 0-2 | 0 | 0 | 0 | 0 | 0 |
| Castus Extract | | | | | | |
| Arginine | 0-2 | 0-2 | 0 | 0 | 0 | 0 |
| Zingiber | 0-5 | 0-5 | 0 | 0 | 0 | 0 |
| officinale | | | | | | |
| Silybum | 0-1 | 0 | 0 | 0 | 0-3 | 0 |
| marianum | | | | | | |
| Extract | | | | | | |
| Apple cider | 0 | 0-40 | 0 | 0 | 0 | 0 |
| vinegar Extract | | | | | | |
| Gynostemma | 0 | 0-1 | 0 | 0 | 0 | 0 |
| pentaphyllum | | | | | | |
| Gymnema | 0 | 0-1 | 0 | 0 | 0 | 0 |
| sylvestre | | | | | | |
| Extract | | | | | | |
| Chromium | 0 | 0-0.001 | 0 | 0 | 0 | 0 |
| Berberine | 0 | 0-2 | 0 | 0 | 0.1-5 | 0 |
| Lagerstroemia | 0 | 0-0.1 | 0 | 0 | 0 | 0 |
| speciosa leaf | | | | | | |
| Extract | | | | | | |
| Grains of | 0 | 0-0.1 | 0 | 0 | 0 | 0 |
| Paradise | | | | | | |
| Extract | | | | | | |
| Curcumin | 0 | 0 | 0-1 | 0 | 0 | 0 |
| Piperine | 0 | 0 | 0-1 | 0 | 0 | 0 |
| Cistanche | 0 | 0 | 0-1 | 0 | 0 | 0 |
| tubulosa | | | | | | |
| Extract | | | | | | |
| Stinging Nettle | 0 | 0 | 0-1 | 0 | 0 | 0 |
| Extract | | | | | | |
| Griffonia | 0 | 0 | 0-1 | 0 | 0 | 0 |
| simplicifolia | | | | | | |
| Extract | | | | | | |
| CoQ10 | 0 | 0 | 0 | 0 | 0.1-2 | 0 |
| (Ubiquinol) | | | | | | |
| NAC (N- | 0 | 0 | 0 | 0 | 0.1-5 | 0 |
| Acetyl- | | | | | | |
| Cysteine) | | | | | | |
| Resveratrol | 0 | 0 | 0 | 0 | 0.1-2 | 0 |
| Pterostilbene | 0 | 0 | 0 | 0 | 0.1-2 | 0 |
| Nicotinamide | 0 | 0 | 0 | 0 | 0-4 | 0 |
| Mononucleotide | | | | | | |
| (NMN) | | | | | | |
| Fisetin | 0 | 0 | 0 | 0 | 0-2 | 0 |
| Milk Thistle | 0 | 0 | 0 | 0 | 0-3 | 0 |
| Extract | | | | | | |
| Quercetin | 0 | 0 | 0 | 0 | 0-3 | 0 |
| Selenium | 0 | 0 | 0 | 0 | 0-0.01 | 0 |
| Trametes | 0 | 0 | 0 | 0 | 0 | 0-1 |
| versicolor | | | | | | |
| Extract | | | | | | |
| Pleurotus | 0 | 0 | 0 | 0 | 0 | 0-1 |
| ostreatus | | | | | | |
| Extract | | | | | | |
| Flammulina | 0 | 0 | 0 | 0 | 0 | 0-1 |
| velutipes | | | | | | |
| Extract | | | | | | |
| Agaricus blazei | 0 | 0 | 0 | 0 | 0 | 0-1 |
| Extract | | | | | | |

Further examples of the composition of the inner core layer 102 include one or more cooperative ingredient combinations that function together for enhanced effects. For example, the composition includes Camellia sinensis Extract plus L-theanine, Caffeine plus L-theanine, GABA plus L-theanine, and/or Magnesium plus L-theanine. For instance, the core composition includes, by weight percentage, 0.5-1% of Camellia sinensis Extract plus 0.1-1% of L-theanine, 0.1-1% of Caffeine plus 0.1-1% L-theanine, 0.1-1% of GABA plus 0.1-1% L-theanine, and/or 0.1-1% of Magnesium plus 0.1-1% L-theanine. Individually, Green Tea Extract is an herbal extract that functions to benefit cognitive functions including memory and attention, L-theanine is an amino acid that functions to reduce stress and anxiety; Caffeine is a natural nootropic found in coffee and tea that functions to increase wakefulness and energy; GABA is an inhibitory neurotransmitter that functions to decrease the stress response and enhancing relaxation; and Magnesium is a mineral nutrient that functions to reduce insomnia. In combination, Camellia sinensis Extract and L-theanine ingredients provide enhanced effects of cognitive alertness, attention, and memory, while reducing stress. In combination, Caffeine and L-theanine ingredients provide improved attention, reaction time, sustained mental clarity, and reduced mental fatigue, In combination, GABA and L-theanine ingredients provide greater relaxation, while reducing stress and anxiety, and compared to each individually alone, together they support improved sleep quality. Another combination, Magnesium and L-theanine together potentiates sleep-promoting effects by enhancing slow brain wave activity indicative of deep, restorative sleep and increasing GABA neurotransmitter and receptor activity.

### 1B. Optional Ingredients and Formulation Flexibility

Variants of the example compositions herein can also include other herbs or minerals to further tailor the effects. For example, magnesium glycinate may be added for additional relaxation benefits, and turmeric extract may be added to provide anti-inflammatory properties. For example, the exact proportions of L-theanine and GABA may be modified within the 0-10% range to optimize efficacy for specific consumer needs. For example, the exact proportions or inclusion of maltitol and/or maltodextrin may be modified to optimize texture.

### 1C. Outer Casing Shell Ingredients

The outer casing layer 101 has a composition including a plurality of ingredients such as, but not limited by weight percentage, of 55-85% of a syrup, 0-25% of maltodextrin (optional for texture), 10-20% of water, 1-7% of stevia leaf extract, 1-5% of pectin, 0-1% of L-malic acid, 0.01-1% of sodium citrate, 0.01-1% of carnauba wax, and 0.01-1% of natural flavors and 0.01-1% of natural color for improved palatability and appearance.

### 2. Mechanisms of Administration

The example nutraceutical gummy 100 and its formulations are formulated for oral administration and ingestion via chewing. For example, a serving (e.g., 1 gummy) of the nutraceutical gummy 100 is provided for oral ingestion. As used herein “provided” or “providing” in the context of administration may include any of selling the nutraceutical gummy 100 in a form that is substantially ready for oral ingestion, prescribing or recommending the nutraceutical gummy 100, or fabricating the nutraceutical gummy 100 from raw starting materials. Upon mastication, the gummy structure allows for the initial release of bioactive ingredients from the inner layer. Bioactives that are suitable for absorption in the oral cavity can thus be readily absorbed through the buccal and sublingual mucosa, providing rapid access to the bloodstream.

Bioactives that are not absorbable in the oral cavity are then swallowed, allowing for further absorption as they travel through the gastrointestinal (GI) tract. This dual-layer composition thus facilitates a unique pathway for both immediate and continued absorption of bioactive compounds, depending on their specific absorption characteristics. Example of absorption characteristics considered include, but are not limited to, (1) molecular weight, where a smaller weights promote oral absorption and larger weights hinder absorption, (2) lipophilicity, where bioactives with moderate lyophilicity allow for crossing of the lipid-rich mucosal membrane, and (3) solubility, where higher solubility promotes oral absorption. The unique dual-layer composition of bioactives allows for optimal absorption of bioactives in the oral cavity and GI tract.

### 3. Fabrication

Fabrication of the nutraceutical gummy 100 in the example formulations preserves the integrity of bioactive compounds through controlled heating and encapsulation steps, which minimizes ingredient degradation and enhances bioavailability. As an example, the nutraceutical gummy 100 can be fabricated in a gummy molding process, a non-limiting example of which is described below. It is to be appreciated that, given this disclosure, one of ordinary skills in the art will be able to readily determine process parameters such as temperatures, durations, and humidity.

### Example Gummy Molding Process

Step 1: The formulation for the inner core layer 102 is prepared by dissolving pectin in deionized water to form a solution, into which sweeteners and active ingredients are incorporated. The solution is then heated and continuously stirred until homogenization.

Step 2: The formulation for the casing shell layer 101 is prepared similarly, with pectin dissolved and sweeteners added, followed by heating and stirring to ensure uniformity.

Step 3: The solution for the inner core layer 102 from Step 1 is then deposited into the center of a preheated mold, followed by depositing into the mold the solution for the casing shell layer 101 from Step 2 to encapsulate the inner core layer 102.

Step 4: The molded gummies are then cooled and dried until reaching a desired moisture content, typically below 20%, which facilitates achievement of a desirable texture of the gummy 100, helps to inhibit degradation of the active ingredients, and helps to avoid over-drying during storage and transit.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A nutraceutical gummy comprising:
a dual-layer configuration including an inner core and, distinct from the inner core, a casing shell fully encasing the inner core, the inner core having a core composition including, by weight percentage, 55-85% of a syrup, 10-20% of water, 1-7% of stevia leaf extract, 1-5% of pectin, the casing shell having a shell composition including, by weight percentage, 55-85% of a syrup, 10-20% of water, 1-7% of stevia leaf extract, and 1-5% of pectin.

2. The nutraceutical gummy as recited in claim 1, wherein the core composition further includes 0.01-1% of L-malic acid, 0.01-1% of sodium citrate, 0.01-1% of natural flavors, 0.01-1% of natural colors, and 0.01-1% of carnauba wax, and the shell composition further includes 0.01-1% of L-malic acid, 0.01-1% of sodium citrate, 0.01-1% of carnauba wax, 0.01-1% of natural flavors, and 0-1% of natural colors.

3. The nutraceutical gummy as recited in claim 1, wherein the core composition further includes 0.1-1% of L-theanine.

4. The nutraceutical gummy as recited in claim 1, wherein the core composition further includes gamma-aminobutyric acid.

5. The nutraceutical gummy as recited in claim 1, wherein the core composition further includes gamma-aminobutyric acid and L-theanine, and a ratio, by weight, of the L-theanine to the gamma-aminobutyric acid is from 5:1 to 1:5 for relaxation or sleep.

6. The nutraceutical gummy as recited in claim 1, wherein the core composition further includes 0.1-3% of a constituent selected from the group consisting of Camellia sinensis, Hericium erinaceus, Cordyceps sinensis, Rhodiola rosea, Lycium barbarum, vitamin B6, vitamin B9, vitamin B12, vitamin D3, Ganodema lucidum extract, and combinations thereof.

7. The nutraceutical gummy as recited in claim 1, wherein the core composition further includes 0.1-1% of a constituent selected from the group consisting of Withania somnifera, Inonotus obliquus, Grifola frondosa, Coriolis versicolor, Lentinula edodes, Passiflora incarnata, Valeriana officinalis, Tremella fuciformis, Wolfporia extensa, Matricaria chamomilla, magnesium glycinate, caffeine, creatine, Alpinia galanga extract, Panax ginseng extract, Schisandra chinensis extract, theobromine, Crocus sativus (saffron) extract, magnesium L-theonate, Conenzyme Q10, N-acetylcysteine, berberine, Resveratrol, Pterostilbene, and combinations thereof.

8. The nutraceutical gummy as recited in claim 1, wherein the core composition further includes 0.1-1% Crocus sativus (Saffron) 0.1-1% Panax Ginseng, 0.1-1% Alpinia Galanga, and 0.1-1% Schisandra chinensis for energy.

9. The nutraceutical gummy as recited in claim 1, wherein the core composition further includes 0.5-1% of Camellia sinensis extract and 0.1-1% of L-theanine for focus and energy.

10. The nutraceutical gummy as recited in claim 1, wherein the core composition further includes 0.1-3% of Camellia sinensis and 0.1-1% of Cordyceps sinensis for energy and endurance.

11. The nutraceutical gummy as recited in claim 1, wherein the core composition further includes 0.1-1% of Cordyceps sinensis and 0.1-1% Rhodiola rosea for stamina.

12. The nutraceutical gummy as recited in claim 1, wherein the core composition further includes 0.1-1% of caffeine and 0.1-1% of L-theanine for focus and energy.

13. The nutraceutical gummy as recited in claim 1, wherein the core composition further includes 0.1-1% of caffeine and 0.1-1% theobromine for focus and energy.

14. The nutraceutical gummy as recited in claim 1, wherein the core composition further includes 0.1-1% of caffeine and 0.1-1% creatine for focus and energy.

15. The nutraceutical gummy as recited in claim 1, wherein the core composition further includes 0.1-1% of caffeine and 0.1-1% Alpinia galanga for energy.

16. The nutraceutical gummy as recited in claim 1, wherein the core composition further include 0.1-1% gamma-aminobutyric acid and 0.1-1% of Passiflora incarnata for relaxation and sleep, or 0.1-1% of Valeriana officinalis and 0.1-1% L-theanine for sleep.

17. The nutraceutical gummy as recited in claim 1, wherein the core composition further includes 0.1-1% of magnesium and 0.1-1% of L-theanine for relaxation or focus.

18. The nutraceutical gummy as recited in claim 1, wherein the core composition further includes at least one of:
0.5-1% of Camellia sinensis and 0.1-1% of L-theanine,
0.1-3% of Camellia sinensis and 0.1-1% of Cordyceps sinensis,
0.1-1% of Cordyceps sinensis and 0.1-1% of Rhodiola rosea
0.1-1% of caffeine and 0.1-1% of L-theanine,
0.1-1% of caffeine and 0.1-1% of theobromine,
0.1-1% of caffeine and 0.1-1% of creatine,
0.1-1% of caffeine and 0.1-1% of Alpinia galanga,
0.1-1% of gamma-aminobutyric acid and 0.1-1% of L-theanine,
0.1-1% of gamma-aminobutyric acid and 0.1-1% of Passiflora incarnata,
0.1-1% of Valeriana officinalis and 0.1-1% of Passiflora incarnata,
0.1-1% of Valeriana officinalis and 0.1-1% of L-theanine, or
0.1-1% of magnesium and 0.1-1% of L-theanine.

19. A method of administration of a nutraceutical gummy, the method comprising:
providing a serving of the nutraceutical gummy for oral ingestion, the nutraceutical gummy having a dual-layer configuration including an inner core and, distinct from the inner core, a casing shell fully encasing the inner core, the inner core having a core composition including, by weight percentage, 55-85% of a syrup, 10-20% of water, 1-7% of stevia leaf extract, 1-5% of pectin, the casing shell having a shell composition including, by weight percentage, 55-85% of a syrup, 10-20% of water, 1-7% of stevia leaf extract, and 1-5% of pectin.

20. The method as recited in claim 19, wherein the oral ingestion includes mastication by chewing, at least a portion of the core composition absorbed through buccal and sublingual mucosa.
